(19) United States
(12) Patent Application Publication
(10) Pub. No.: US 2023/0061887 A1
(43) Pub. Date: Mar. 2, 2023

(54) ROD CUTTER

(71) Applicant: ln'Tech Medical SAS, Rang-du-Fliers (FR)

(72) Inventors: MARIUS JOLY, VIEIL-MOUTIER (FR); STEPHANE HAUDIQUET, SORRUS (FR)

(73) Assignee: ln'Tech Medical SAS, Rang-du-Fliers (FR)

(21) Appl. No.: 17/697,360

(22) Filed: Mar. 17, 2022

Related U.S. Application Data
(60) Provisional application No. 63/237,723, filed on Aug. 27, 2021.

(51) Int. Cl. A61B 17/88

(52) U.S. Cl. CPC A61B 17/8863; A61B 2017/00407

(57) ABSTRACT

A rod cutter that has a ratcheting mechanism that allows incremental, stepped forces applied to the rod being cut. The ratcheting mechanism employs multiple pawls to retain the rod cutter in stepped positions during the cutting process.

### RELATED APPLICATIONS

This application claims priority to U.S. Application Ser. No. 63/237,723, filed on 27 Aug. 2021.

### BACKGROUND OF THE INVENTION

The present invention is directed toward rod cutters, particularly rod cutters that can be used during surgery.

Orthopedic surgeries, particularly those involving the spine, require tools and instruments having very specific features and operating features. For example, the instruments must be made of biocompatible materials, as they will be in contact with various tissues, e.g. muscles, bones, organs, etc. Further, besides being in contact with various tissues, the devices must be designed to work in and around these tissues, such around the spinal cord, without causing any undue damage to the area.

Tools, such as rod cutters, are often used in and around the spinal area and require a combination of flexibility and strength, as they are used to cut hard materials such as bone or metal. Such metals can include titanium or chrome cobalt. Also, the tools must be capable of very precise movements while still providing the necessary strength to carry out procedures.

As an example, rod cutters are used for procedures for realigning vertebrae, in the case of treatment of a herniated disc. A rod is placed, fitted, and cut in situ. The standard rod used in such an operation was a titanium rod a diameter of 4 mm. However, as the population has evolved and more and more of the population is overweight, stronger materials, such as chrome cobalt are needed to properly carryout such procedures. Current pliers and cutting devices do not have the necessary specifications to carry out such procedures, as the effort to cut these rods is too high with current devices.

Thus, there is a need of a rod cutter capable of cutting chrome-cobalt rods with a diameter of at least 4 mm, while adapting to the many constraints inherent in surgery: choice of materials, size, strength, flexibility and resistance, as well as ergonomic issues.

### SUMMARY OF THE INVENTION

The present invention is directed towards a rod cutter that is capable of providing a force sufficient to cut a 4 mm chrome-cobalt rod in situ. The rod cutter is capable of being operated with a single hand.

The present invention further provides a rod cutter that can apply the necessary force for the cutting process that increases as needed during the cutting process. The rod cutter includes a reducer in combination with a ratchet gear that allows for the rod cutter to be operated with one hand.

### BRIEF DESCRIPTION OF THE DRAWINGS

FIG. 1 is a perspective view of a rod cutter according to the present invention.

FIG. 2 a side view of the rod cutter of FIG. 1.

FIG. 3 is a front view of the rod cutter of FIG. 1.

FIG. 4 an exploded view of the rod cutter of FIG. 1.

FIG. 5 is a sectional view of the rod cutter of FIG. 1 taken along the line 5-5 of FIG. 1.

FIG. 5A is a close-up view of the gear shown in FIG. 5.

FIG. 6 demonstrates an alternate rod cutter according to the present invention.

FIG. 7 demonstrates the rod cutter of the present invention being gripped by a user.

FIG. 8 demonstrates an up-close view of the spinal area with the rod cutter being used to initially cut a rod.

FIG. 9 depicts a front view of the rod cutter shown in FIG. 8.

FIG. 10 demonstrate the rod cutter further cutting through the rod of FIG. 8.

FIG. 11 demonstrates the rod cutter cutting through the rod of FIG. 10.

FIG. 12 demonstrates the rod cutter returning to an open position.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

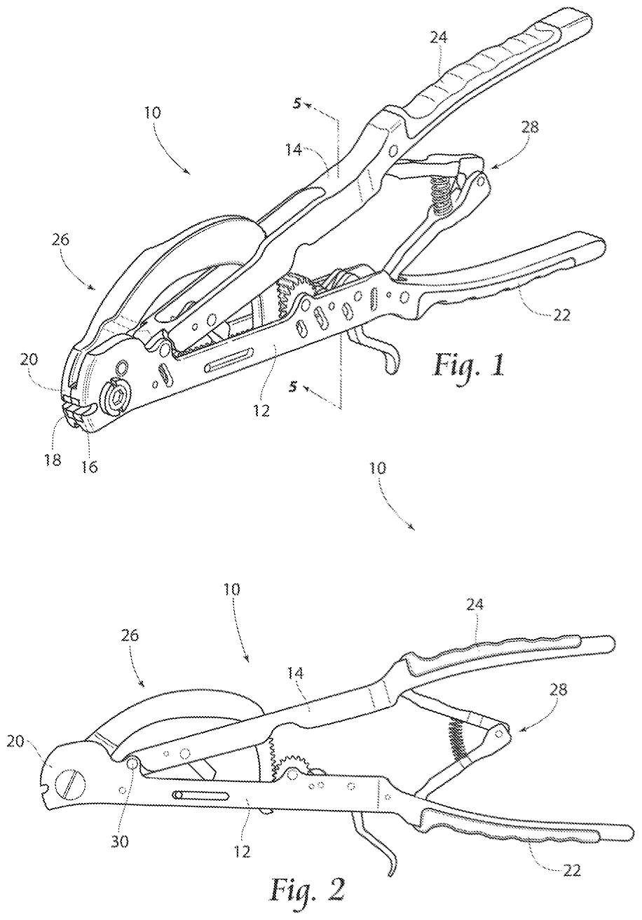

FIG. 1 is a perspective view of a rod cutter 10 according to the present invention. The rod cutter 10 comprises a first arm 12 and a second arm 14 hinged to one another. Each of the arms 12 and 14 has an associated cutting jaw 16 and 18 located at a front end 20 of the rod cutter 10. The arms 12 and 14 also have a respective handle portion 22 and 24 that allows the user to properly grip the rod cutter 10.

Still referring to FIG. 1, the rod cutter also comprises a ratcheting system or mechanism 26 and a return system 28. As will be discussed in further detail below, the ratcheting system 26 allows for the user to apply a gripping force with a singular hand to the rod cutter 10 that is sufficient for cutting a rod, particularly as the necessary force increases during the cutting process. The rod cutter 10 allows for the increasing delivery of force during the entire process.

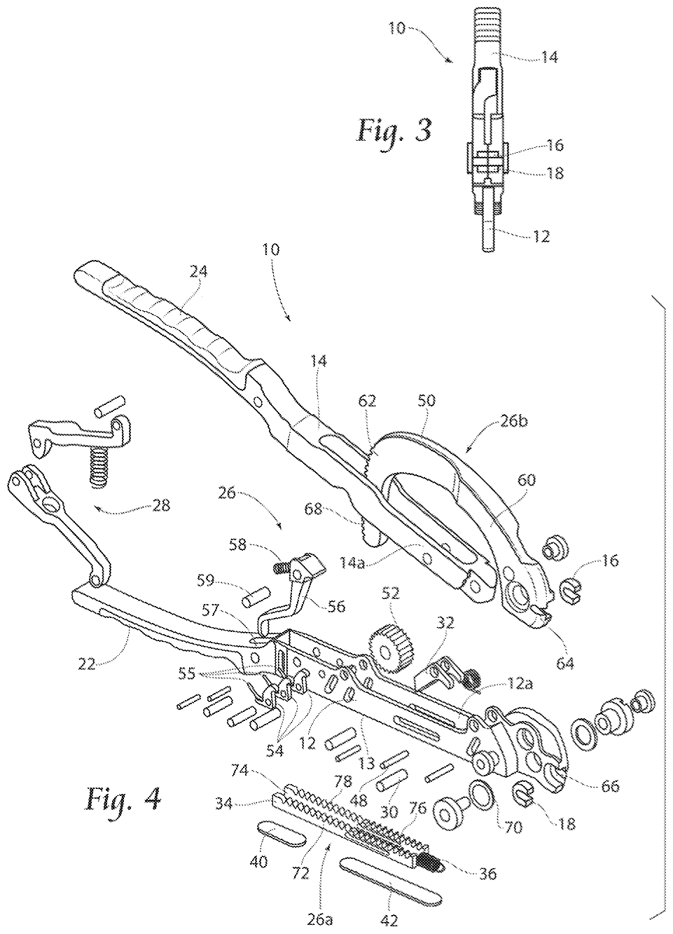

FIG. 2 provides a side view of the rod cutter 10. As noted, the arms 12 and 14 are hinged to one another with a pin 30 or similar device that allows for the arms 12 and 14 to pivot to one another. The arms 12 and 14 are also connected to one another by use of the ratcheting system 26 as well as the return system 28. The arrangement of the two arms 12 and 14 is a linear arrangement along a central axis (see FIG. 3) that provides for a smooth and efficient transfer of the force from the user's movements to the rod that will be covered.

Now referring to FIG. 4, an exploded view of the rod cutter 10 is shown. The ratcheting system 26 is shown, generally comprising a first section 26a and a second section 26b. The first section 26a is generally positioned on the first arm 12, which can be referred to as the fixed arm, and the second section 26b generally positioned and aligned with on the second arm 14, also referred to as the moving arm. More specifically, the first section 26a is positioned within an opening 12a within the first arm 12, and the second section 26b is positioned within an opening 14a located in the second arm 14. It should be noted that the second section 26b is hinged to the first arm 12. As will be evident in the discussion below, the two sections 26a and 26b are also positioned and attached to each of the other arms, as well. The opening 12a has a bottom surface 13 with an aperture 15 located in the bottom surface 13.

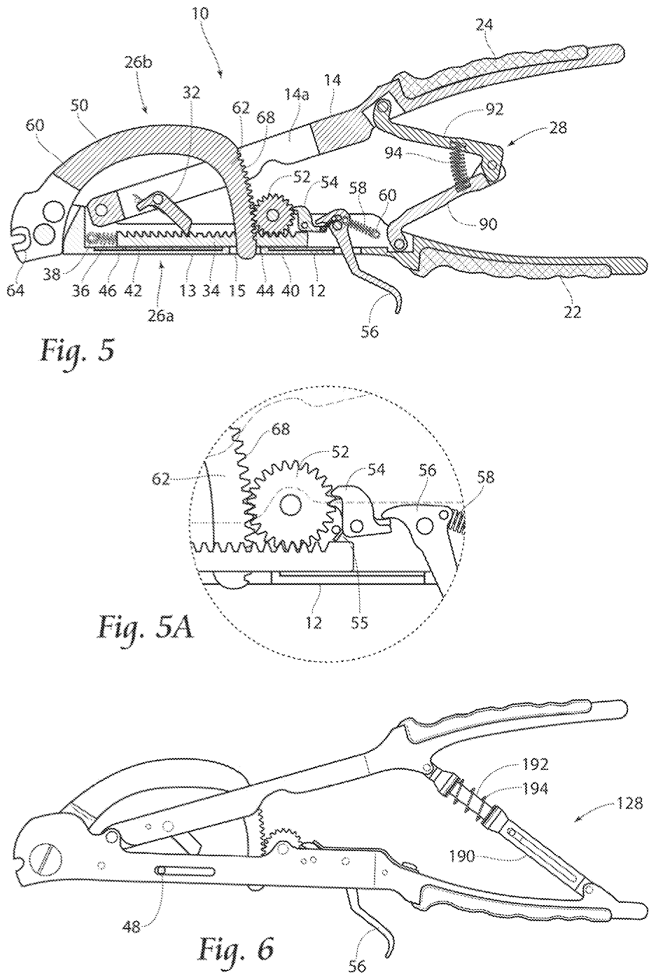

Referring further to FIG. 4 (and FIG. 5) the first section comprises a front pawl 32 that interacts with a linear rack 34. The linear rack 34 is attached to the first arm 12 with the use of a spring 36 attached to the first arm 12 with a pin 36 or other fastening mechanism. The linear rack 34 is also positioned within the opening 12a so that it rests on friction pads 40 and 42, which are preferably positioned within recesses 44 and 46 located in the bottom surface 13. The friction pads 40 and 42 facilitate the movement of the linear rack 34 as it glides through the ratcheting process. Preferably, the friction pads 40 and 42 are made of a material having a lower coefficient of material than the rod cutter 10 (and the first arm 12). For example the rod cutter 10 and arm 12 may be made of stainless steel or similar material, while the friction pads 40 and 42 may be made of a polymer material, such as polyether ether ketone (PEEK polymer) or other similar material to assist in reducing friction. Further to assist in the even movement of the linear rack 34, alignment pin or pins 48 may be positioned to intersect the linear rack and sit within slots located in the opening 12a.

Still referring to FIGS. 4 and 5 and also FIG. 5A, the second section 26b of the ratcheting mechanism 26 is shown having an arm 50 and a gear 52. The gear 52 engages with the pawls 54 that are pivotally connected to the arm 12. Preferably there are at least three pawls 54 arranged at differing angles, which reduces the pawl pitch and increases the ratcheting performance of the device 10. As shown particularly in FIG. 5A, the pawls intersect the gear 52 at various positions to reduce the standard ratcheting pitch. The pawls 54 are further held in place with springs 55 linked to a lever 56 located within an opening 57 in the arm. The lever 56 is also connected to the arm 12 with a spring 56 and pin 59 or similar fastening mechanism.

Referring again to the arm 50, it comprises a first section 60 and a second section 62. The first section 60 is pivotally connected to the first arm 12, with the first section 60 providing a support area 64 for the cutting jaw 16. The support area 64 is generally aligned with a support area 66 located on the arm 12 for the cutting jaw 18. The second section 62 is located relatively perpendicular to the first section 60, with the second section 62 having a serrated edge 68. The second section 62 is positioned that it will pass through the aperture 15 located in the bottom 13 of the arm 12 when force is applied by the user to the handle portions 22 and 24. Preferably, the linear rack 34 comprises opposing sides 70 and 72, with a pathway 74 there between. The second section 62 is preferably positioned within the pathway 74.

The ratcheting mechanism 26 is further designed to carry out the ratcheting procedure according to the present invention with the design of the linear rack 34. The linear rack 34 has a front section 76 interacting with the pawl 32 and a rear section 76 interacting with the gear 52. Preferably each section 76 and 78 is serrated for its intended use in facilitating the ratcheting action. For example, the front section 76 retains the pawl 32 in a particular position, while the rear section 78 allows for interaction from both the serrated edge 68 and the gear 52.

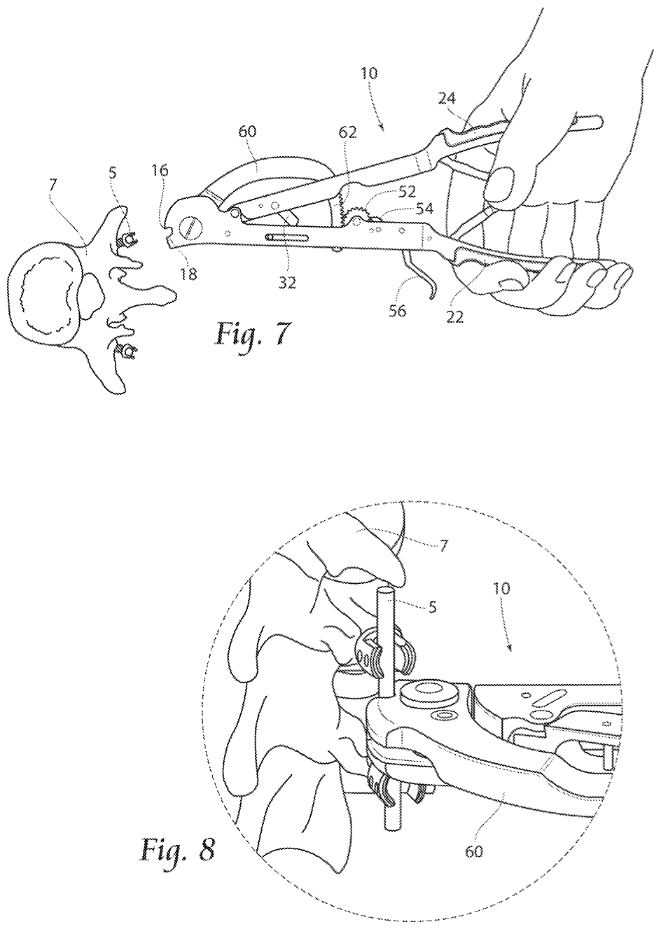

Because of the ratcheting mechanism arrangement, the user can provide the requisite amount of force for cutting a rod in situ with a tool that can function in a realistic manner during a surgical procedure. The user must be able to apply at least a force of 13700 N and preferably 15000 N to cut a rod as described within the area. Typically, an incision is less than 4 inches (˜102 mm) in diameter that an instrument has to be inserted, and likely less than 3.75 inches (95 mm). The present rod cutter 10 is designed with these specifications, with the racket system 26 balancing the output stroke of the user versus the input stroke of the rod cutter to ensure that the rod cutter can accomplish the procedure with a single hand and force at the output to cut the rod. Further, the arrangement of the racket system allows for the force of the user to be increased, e.g. multiplied, and applied to the rod being cut. It is contemplated that the rod cutter will apply a force to a rod that is several times greater than the input force (e.g. more than 40 times the input force). This arrangement, described above, will be further demonstrated with respect to FIGS. 7-12 below.

The rod cutter 10 also has a return system 28, which allows for the rod cutter 10 to return to the open position once a procedure is finished, or if the rod cutter 10 needs to be repositioned. FIGS. 4 and 5 show the return system, which generally comprises to scissor arms 90 and 92 connected to each of the arms 12 and 14. The arms 90 and 92 are connected at a pivot, with a spring 94 biasing the arms 90 and 92. Once the lever 56 is activated, the spring 94 will return the rod cutter 10 to an open position.

FIG. 6 demonstrates an alternate return system 123. The system 128 generally consists of two telescoping rods 190 and 192 connected to a respective arm 12 and 14. Once or more concentric springs 194 are located on the internal rod 192 and are compressed as the rod cutter 10 is operated. As with the return system 28, the return system 128 is allowed to return to an open position once the lever 56 is activated.

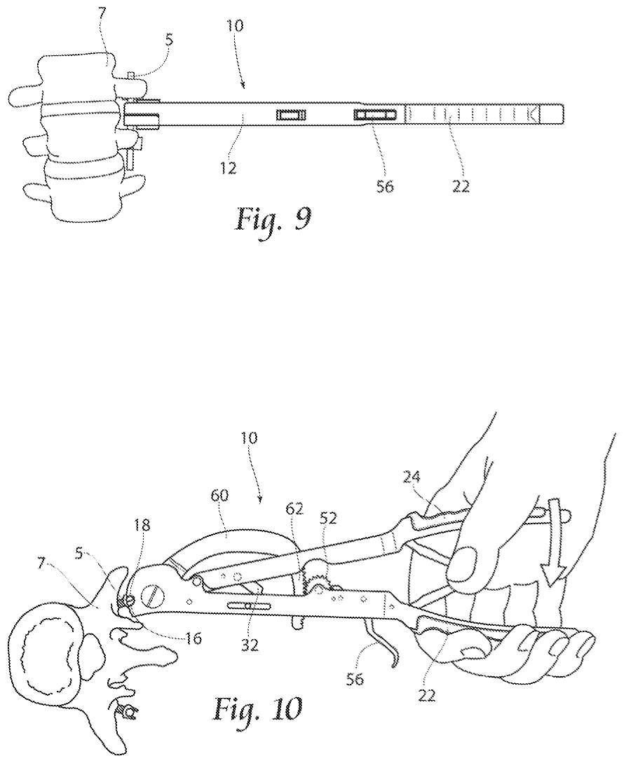

The rod cutter 10 described above will be demonstrated in FIGS. 7-12. The user grabs the rod cutter 10 by the handle portion 22 and 24 and moves it inwardly towards a rod 5 attached to a vertebrae 7 (FIG. 7). The cutting jaws 16 and 18 are positioned around the rod 5 (FIG. 8 and FIG. 9).

As shown in FIG. 10, the user squeezes on the handle portions 22 and 24, thereby having the arm 14 engaging the pawl 32. The interaction moves the linear rack to thereby move the gear 52, which engages the serrated edge 68, resulting in movement of the jaw 16.

The cutting jaws 16 and 18 will be moved closer to one another and to engage the rod 5. The linear rack 34 will also be moved by the gear 52, causing the front pawl 32 to move to another forward position. The position will be maintained until a further force is applied by the user to the rob cutter 10.

To further assist in the cutting process, as noted above, the pawls 54 are designed with each of the individual pawls 54 having a different angle of locking teeth, essentially dividing the standard ratcheting pitch by three. That is, the pawls 54 are angled in such a manner that only one of the pawls 54 will engage with the gear 52 at one time, further enhancing the translation of the user's force to the ratcheting process. It should be understood that more or fewer pawls 54 may be used, with each of them also positioned at a different angle position.

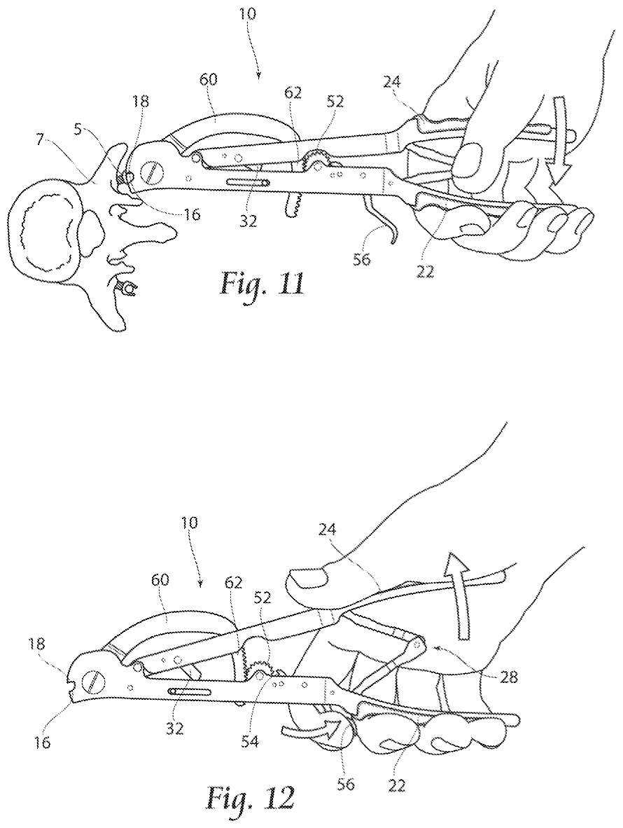

The user can further grip the handle portions 22 and 24 to bring the cutting jaws 16 and 18 further together and eventually cut through the rod 5 (FIG. 11). Once finished, the user can apply force to the lever 56, which will disengage the gear 52 from the serrated edge 68. The return system 28 will move the arms 12 and 14 outwardly from one another. The front pawl 32 will be released and the rod cutter 10 is returned to the open position.

Thus, the present invention provides an efficient rod cutter that addresses several issues with current cutting devices. The rod cutter can efficiently operate within the spatial constraints of the surgical procedure. The device allows sufficient force to be applied by the user with a single hand to cut standard rods use in surgical procedures. The present invention also allows for the user to have a more controlled process, as the ratcheting system allows for the cutting jaws to be retained in several stepped positions between a full open and full closed (i.e. the rod is cut) position. As noted, the present invention is designed to cut a standard rod of at least 4 or 5 mm. However, it is contemplated that the rod cutter could be used on rod cutters of smaller sizes or larger sized rods, e.g. rods of 6 mm or greater. Provided that the rod cutter functions as discussed herein, such a rod cutter would fall within the scope of the present invention.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I/We claim:

1. A rod cutter comprising:
a first arm;
a second arm hinged to said first arm;
a first cutting jaw located on said first arm;
a ratcheting mechanism connected to said first and said second arms and said cutting jaw, said ratcheting mechanism moving said cutting jaw in an inwardly stepped fashion.

2. The rod cutter of claim 1 further comprising:
a second cutting jaw on said second arm, said ratcheting mechanism moving said cutting jaws in a stepped fashion.

3. The rod cutter of claim 1, wherein said ratcheting mechanism further comprises
a pawl for retaining said cutting jaw in a stepped position.

4. The rod cutter of claim 3 further comprising
a release mechanism for moving said cutting jaw outwardly from said stepped position.

5. The rod cutter of claim 3 wherein said ratcheting mechanism further comprises:
a serrated track located on said first arm, said serrated track interacting with said pawl to retain said cutting jaw in said stepped position.

6. The rod cutter of claim 5 wherein said ratcheting mechanism can retain said cutting jaw in multiple stepped positions.

7. The rod cutter of claim 6 wherein said ratcheting mechanism further comprises:
a serrated arm connected to said second arm; and
a gear interacting with said serrated arm and said serrated track to move said cutting jaw inwardly.

8. A rod cutter comprising:
a first arm;
a second arm hinged to said first arm;
a first cutting jaw located on said first arm;
a second cutting jaw located on said second arm;
a ratcheting mechanism connected to said first and said second arms and said cutting jaws, said ratcheting mechanism moving said cutting jaws in an inwardly stepped fashion; and
a release mechanism for moving said cutting jaws outwardly.

9. The rod cutter of claim 8 wherein said ratcheting mechanism further comprises:
a pawl for retaining said cutting jaw in a stepped position; and
a serrated track located on said first arm, said serrated track interacting with said pawl to retain said cutting jaw in said stepped position.

10. The rod cutter of claim 9 wherein said ratcheting mechanism further comprises:
a serrated arm connected to said second arm;
a gear interacting with said serrated arm and said serrated track to move said cutting jaw inwardly; and
a plurality of second pawls interacting with said gear.

11. A rod cutter comprising:
a first rod cutter arm;
a second rod cutter arm hinged to said first arm;
a first cutting jaw located on said first arm;
a ratcheting mechanism connected to said first and said second arms and said cutting jaws, said ratcheting mechanism moving said cutting jaw in an inwardly stepped fashion; said ratcheting mechanism comprising:
a pawl for retaining said cutting jaw in a plurality of stepped positions;
a serrated track located on said first arm, said serrated track interacting with said pawl to retain said cutting jaw in said stepped position;
a ratcheting arm having a first section pivotally connected to said second arm and a second serrated section; and
a gear interacting with said serrated arm and said serrated track to move said cutting jaw inwardly.

12. The rod cutter of claim 11 further comprising:
a return system for moving said cutting jaw outwardly.

13. The rod cuter of claim 12 wherein the return system further comprises
a release mechanism for moving said cutting jaws outwardly.

14. The rod cutter of claim 13 wherein the release mechanism comprises:
a lever movable between and engaged position with said gear to a disengaged position, wherein said cutting jaws are retained in one of said stepped positions when said lever is in said engaged position and wherein said cutting jaws are capable of moving outwardly when in said disengaged position.

15. The rod cutter of claim 14 wherein the return system further comprises:
a first scissor arm pivotally connected to said first rod cutter arm;
a second scissor arm pivotally connected to said second rod cutter arm;
said first and second arm pivotally connected to one another; and
biasing means for pushing said rod cutter arms outwardly when said lever is in said disengaged position.

16. The rod cutter of claim 11 wherein said ratcheting mechanism can retain said cutting jaw in multiple stepped positions.

17. The rod cutter of claim 16 wherein said cutting jaw applies a force of at least 13700 N to the rod.

18. The rod cutter of claim 17 wherein said cutting jaw applies a force of at least 15000 N to the rod.

19. The rod cutter of claim 11 wherein said ratcheting arm first section is relatively perpendicular to said ratcheting arm second section.

20. A method of using a rod cutter according to claim 11.
